(19) United States
(12) Patent Application Publication
(10) Pub. No.: US 2023/0288360 A1
(43) Pub. Date: Sep. 14, 2023

(54) GAS SENSOR, ELECTRONIC APPARATUS, AND METHOD OF SENSING GAS

(71) Applicant: Sharp Semiconductor Innovation Corporation, Tenri City (JP)

(72) Inventors: Toshiya FUJIYAMA, Tenri City (JP); Noboru TAKEUCHI, Tenri City (JP); Daiki NARUSE, Tenri City (JP)

(21) Appl. No.: 18/119,110

(22) Filed: Mar. 8, 2023

(30) Foreign Application Priority Data
Mar. 10, 2022 (JP) 2022-037445

(51) Int. Cl. G01N 27/12; G01N 33/00

(52) U.S. Cl. CPC G01N 27/122; G01N 27/125; G01N 33/0027; G01N 33/0067

(57) ABSTRACT

A gas sensor includes: a sensor surface on which a metal-oxide film grows; a detection unit configured to detect a change in a resistance value of the metal-oxide film; and a computation unit configured to compute a quantity of reducing gas in measurement-target air based on a result of the detection by the detection unit, wherein the gas sensor operates in a first mode in which the gas sensor stands by with standby air, which differs from the measurement-target air, being in contact with the sensor surface immediately after the gas sensor is activated and in a second mode that follows the first mode and in which, the detection unit detects a change in the resistance value, and the computation unit computes the quantity of the reducing gas based on the result of the detection, with the measurement-target air being in contact with the sensor surface.

### CROSS-REFERENCE TO RELATED APPLICATION

The present application claims priority from Japanese Application JP2022-037445, the content of which is hereby incorporated by reference into this application.

### BACK GROUND OF THE INVENTION

### 1. Field of the Invention

The present invention relates to gas sensors, electronic apparatuses, and methods of sensing a gas, all involving use of a metal-oxide film.

### 2. Description of the Related Art

Semiconductor gas sensors sense gas by detecting a change in the resistance value of a metal-oxide film caused by an oxidation-reduction reaction upon the metal-oxide film coming into contact with the gas.

PCT International Application Publication No. WO2019/220741 describes a gas detection device that detects gas by processing, using a digital information processing device, an output of a metal-oxide semiconductor gas sensor that decreases the resistance value thereof in a reducing gas and comparing a result with a comparative value for gas detection. In the gas detection device described in PCT International Application Publication No. WO2019/220741, the digital information processing device extracts data representing the resistance value of the gas sensor in air from the output of the gas sensor, and the comparative value is generated such that the higher the resistance value of the gas sensor in air, the larger the ratio of the resistance value in air and the resistance value corresponding to the comparative value.

### SUMMARY OF THE INVENTION

Observations by the inventors of the present invention meanwhile indicate that the semiconductor gas sensor exhibits undesirable, low detection sensitivity for some time after the sensor is activated because the sensor is not capable of properly detecting gas when the metal-oxide film has not sufficiently grown. As an example, the sensor may detect no gas at all if the sensor is designed to do so by converting the quantity of a TVOC (total volatile organic compound) to an equivalent quantity of eCO₂. PCT International Application Publication No. WO2019/220741 does not discuss any such problems of the detection sensitivity that could occur after the activation of the sensor.

The present invention, in an aspect thereof, has an object to improve the post-activation detection sensitivity of a gas sensor that uses a metal-oxide film.

To achieve this object, the present invention, in one aspect thereof, is directed to a gas sensor including: a sensor surface on which a metal-oxide film grows; a detection unit configured to detect a change in a resistance value of the metal-oxide film; and a computation unit configured to compute a quantity of reducing gas in measurement-target air based on a result of the detection by the detection unit, wherein the gas sensor operates in a first mode in which the gas sensor stands by with standby air, which differs from the measurement-target air, being in contact with the sensor surface immediately after the gas sensor is activated and in a second mode that follows the first mode and in which, the detection unit detects a change in the resistance value, and the computation unit computes the quantity of the reducing gas based on the result of the detection, with the measurement-target air being in contact with the sensor surface.

The present invention, in one aspect thereof, can improve the post-activation detection sensitivity of a gas sensor that uses a metal-oxide film.

### BRIEF DESCRIPTION OF THE DRAWINGS

FIG. 1 is a cross-sectional side view of a gas sensor in accordance with Embodiment 1 of the present invention.

FIG. 2 is a functional block diagram of the gas sensor in accordance with Embodiment 1 of the present invention.

FIG. 3 is a flow chart representing an operation of the gas sensor in accordance with Embodiment 1 of the present invention.

FIG. 4 is a diagram illustrating the growth of a metal-oxide film in the gas sensor in accordance with Embodiment 1 of the present invention.

FIG. 5 contains a graph representing a relationship between the time elapsed since the gas sensor is activated and the thickness of the metal-oxide film under different aerial conditions on the left-hand side and a graph representing a relationship between the time elapsed since the gas sensor is activated and the sensitivity of the gas sensor under different aerial conditions on the right-hand side.

FIG. 6 is a diagram illustrating an example of standby air supply in the gas sensor in accordance with Embodiment 1 of the present invention.

FIG. 7 is a functional block diagram of a gas sensor in accordance with Embodiment 2 of the present invention.

FIG. 8 is a flow chart representing an operation of the gas sensor in accordance with Embodiment 2 of the present invention.

FIG. 9 is a graph representing a relationship between the time elapsed since the gas sensor is activated and a correction coefficient under different aerial conditions.

FIG. 10 is a cross-sectional side view of an electronic apparatus in accordance with Embodiment 3 of the present invention.

FIG. 11 is a functional block diagram of the electronic apparatus in accordance with Embodiment 3 of the present invention.

FIG. 12 is a flow chart representing an operation of the electronic apparatus in accordance with Embodiment 3 of the present invention.

FIG. 13 is a diagram illustrating an example of standby air supply in the electronic apparatus in accordance with Embodiment 3 of the present invention.

FIG. 14 is a diagram illustrating an example of measurement target supply in the electronic apparatus in accordance with Embodiment 3 of the present invention.

FIG. 15 is a cross-sectional side view of an electronic apparatus in accordance with Embodiment 4 of the present invention.

FIG. 16 is a cross-sectional top view of the electronic apparatus in accordance with Embodiment 4 of the present invention.

FIG. 17 is a flow chart representing an operation of the electronic apparatus in accordance with Embodiment 4 of the present invention.

FIG. 18 is a diagram illustrating an example of standby air supply in the electronic apparatus in accordance with Embodiment 4 of the present invention.

FIG. 19 is a diagram illustrating an example of measurement-target air supply in the electronic apparatus in accordance with Embodiment 4 of the present invention.

### DETAILED DESCRIPTION OF THE INVENTION

### Embodiment 1

The following will describe Embodiment 1 of the present invention in detail. A gas sensor 1 in accordance with the present embodiment senses a reducing gas in air. The gas sensor 1 may sense any material including TVOC and other various reducing gases. The gas sensor 1 may be installed in, for example, an air purifier, an air conditioner, or any other apparatus.

### Structure of Gas Sensor

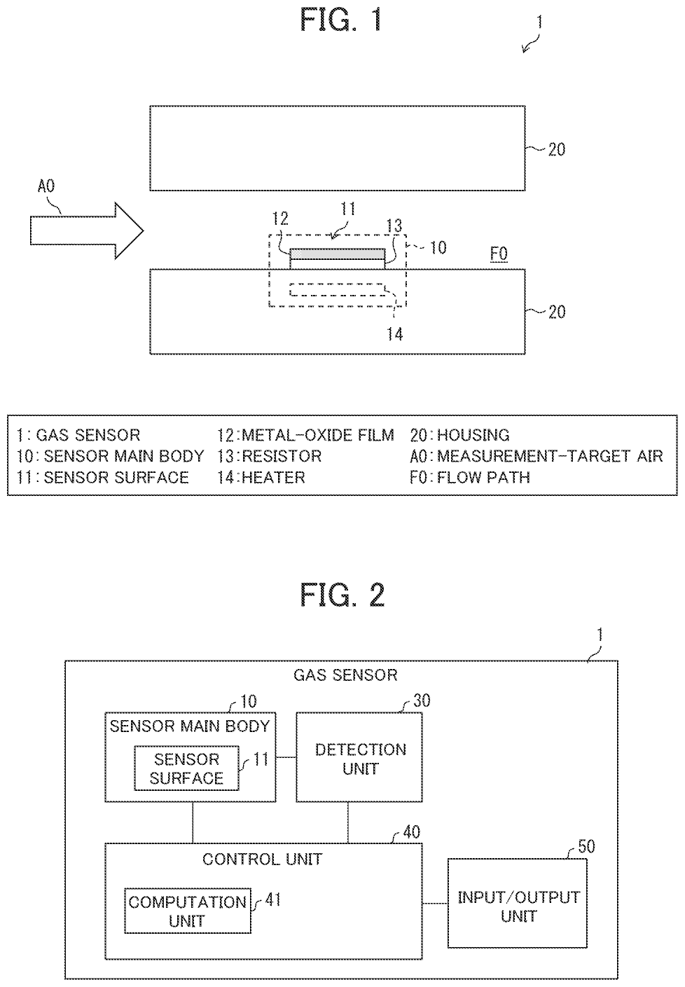

FIG. 1 is a cross-sectional side view of the gas sensor 1. FIG. 2 is a functional block diagram of the gas sensor 1. The gas sensor 1 includes a sensor main body 10, a housing 20, a detection unit 30, a control unit 40, and an input/output unit 50.

The sensor main body 10 includes: a sensor surface 11 on which a metal-oxide film 12 grows; and a heater 14 for promoting the growth of the metal-oxide film 12. The sensor surface 11 is structured so as to enable the metal-oxide film 12 to grow on a resistor 13 when heated by the heater 14. The heating temperature is not limited in any particular manner and may be, for example, from a few hundred degrees Celsius to a few thousand degrees Celsius.

The metal-oxide film 12 may be a publicly known metal-oxide film used in a semiconductor gas sensor.

The housing 20 provides a flow path F0 where the sensor surface 11 is disposed. To measure measurement-target air A0, measurement-target air A0 flows through the flow path F0, thereby being fed to the sensor surface 11.

The detection unit 30 is a circuit for detecting a change in the resistance value of the metal-oxide film 12 and may be built around, for example, an AFE (analog front end).

The control unit 40 collectively controls the processes performed by various members and units of the gas sensor 1. The control unit 40 includes a computation unit 41. The computation unit 41 computes the quantity of reducing gas in measurement-target air A0 on the basis of results of the detection performed by the detection unit 30. The computation unit 41 may convert the quantity of reducing gas to an equivalent quantity of carbon dioxide (eCO₂) for computation.

The input/output unit 50 receives inputs of various information and makes outputs of, for example, results of the computation by the computation unit 41. The input/output unit 50 may include a means for communications with another device, apparatus, or unit and may include publicly known input means such as a keyboard, a mouse, and a touch panel and publicly known output means such as a display device and a speaker.

### Overview of Operation of Gas Sensor

The gas sensor 1 is a semiconductor gas sensor. The metal-oxide film 12 can grow on the sensor surface 11. As measurement-target air A0 containing reducing gas is supplied to the sensor surface 11, the reducing gas comes into contact with the metal-oxide film 12, causing an oxidation-reduction reaction which changes the resistance value of the metal-oxide film 12. The detection unit 30 detects this change in the resistance value of the metal-oxide film 12. The computation unit 41 then computes the quantity of the reducing gas in measurement-target air A0 on the basis of results of this detection, to sense the reducing gas.

FIG. 4 is a diagram illustrating the growth of the metal-oxide film 12 in the gas sensor 1. As shown in the left-hand side of FIG. 4, there is almost no metal-oxide film 12 grown on the sensor surface 11 immediately after the gas sensor 1 is activated. As described here, immediately after the gas sensor 1 is activated, the metal-oxide film 12 is present in a limited amount and therefore only reacts in a limited amount with the reducing gas, which makes it difficult to sense the reducing gas.

As the gas sensor 1 is activated, and the heater 14 heats up, the metal-oxide film 12 gradually grows on the sensor surface 11 as shown in the right-hand side in FIG. 4. The gas sensor 1 hence becomes properly ready to sense the reducing gas.

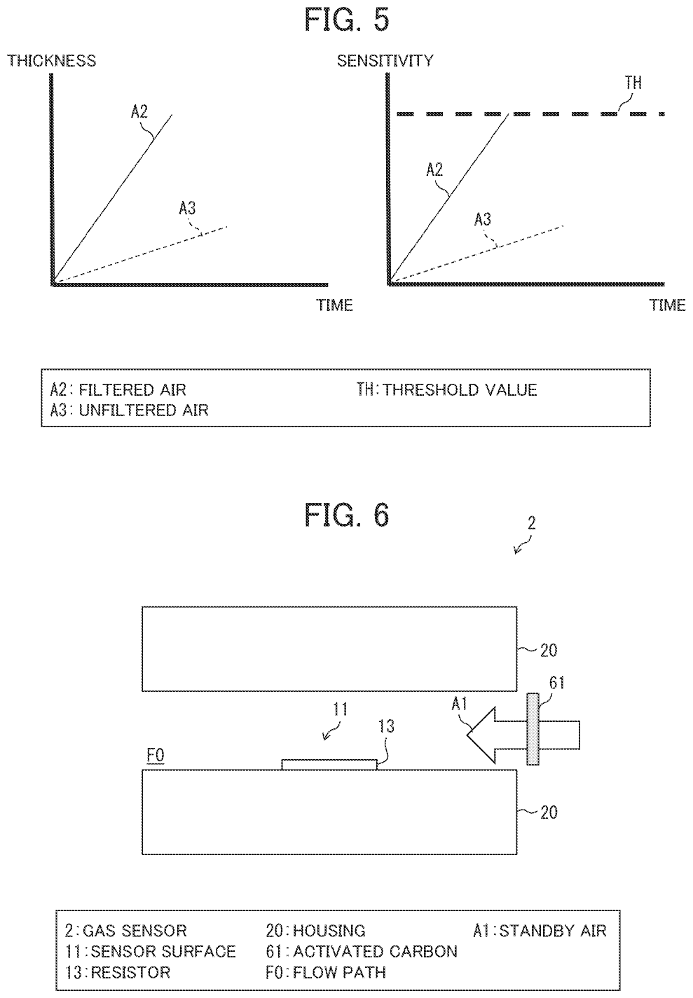

The growth rate of the metal-oxide film 12 is affected by the quality of the air surrounding the sensor surface 11. The graph in the left-hand side of FIG. 5 represents a relationship between the time elapsed since the gas sensor 1 is activated and the thickness of the metal-oxide film 12 for a case where the sensor surface 11 is surrounded by filtered air A2 and for a case where the sensor surface 11 is surrounded by unfiltered air A3. The metal-oxide film 12 exhibits a higher growth rate when the sensor surface 11 is surrounded by filtered air A2 than when the sensor surface 11 is surrounded by unfiltered air A3, as shown in the left-hand side of FIG. 5.

As described earlier, the gas sensor 1 senses reducing gas through a reaction of the metal-oxide film 12 and for this reason has sensitivity that varies with the thickness of the metal-oxide film 12. The graph in the right-hand side of FIG. 5 represents a relationship between the time elapsed since the gas sensor is activated and the sensitivity of the gas sensor for a case where the sensor surface 11 is surrounded by filtered air A2 and for a case where the sensor surface 11 is surrounded by unfiltered air A3. The sensitivity of the gas sensor 1 reaches a prescribed threshold value TH more quickly when the sensor surface 11 is surrounded by filtered air A2 than when the sensor surface 11 is surrounded by unfiltered air A3, as shown in the left-hand side of FIG. 5. The threshold value TH is specified so as to deliver sufficient sensitivity to sense reducing gas.

The gas sensor 1 in accordance with the present embodiment therefore operates in a first mode in which the gas sensor 1 stands by with the sensor surface 11 being in contact with standby air A1, which differs from measurement-target air A0, immediately after the gas sensor 1 is activated and in a second mode that follows the first mode and in which the detection unit 30 detects a change in the resistance value of the metal-oxide film 12, and the computation unit 41 computes the quantity of reducing gas on the basis of results of this detection, with the sensor surface 11 being in contact with measurement-target air A0.

Standby air A1 differs from measurement-target air A0 and preferably promotes the growth of the metal-oxide film 12 better than measurement-target air A0. Standby air A1 may be, for example, filtered air. Standby air A1 preferably contains TVOC that is equivalent to approximately 400 ppm eCO₂.

Hence, immediately after the gas sensor 1 is activated, the gas sensor 1 stands by with standby air A1 being in contact with the sensor surface 11, which facilitates the growth of the metal-oxide film 12 on the sensor surface 11. That in turn enables improving the post-activation detection sensitivity of the sensor. In particular, if standby air A1 is filtered air, the growth of the metal-oxide film 12 on the sensor surface 11 is suitably facilitated. Even when the computation unit 41 computes the quantity of reducing gas through conversion to an equivalent quantity of carbon dioxide (eCO₂), the presence/absence of reducing gas is readily checked because of the improved sensitivity.

### Details of Operation of Gas Sensor

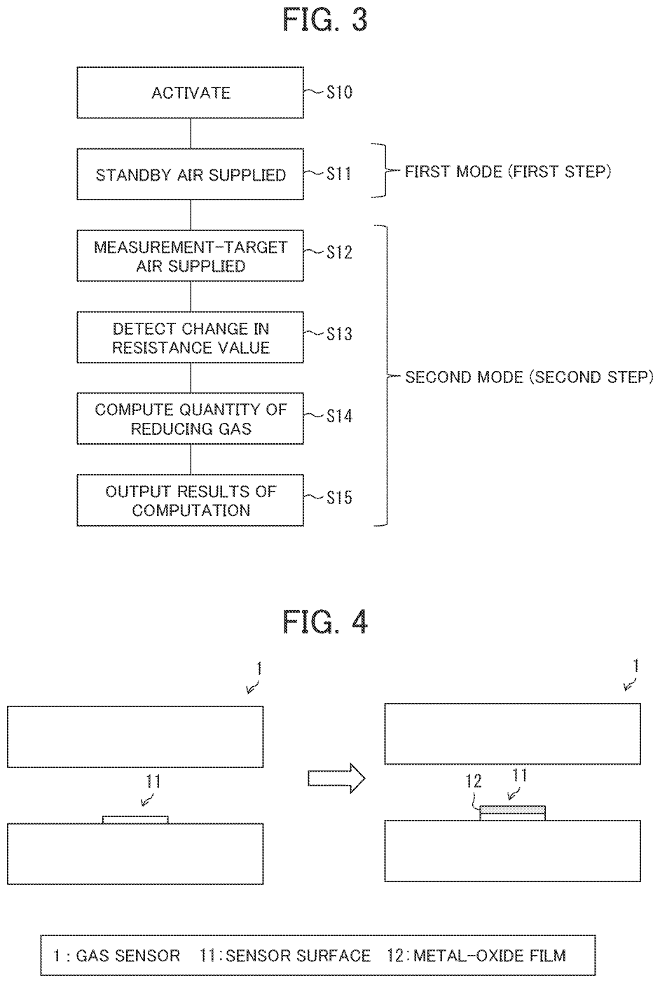

FIG. 3 is a flow chart representing an operation (method of sensing a gas) of the gas sensor 1.

In step S10, the gas sensor 1 is activated, and the control unit 40 starts control. The heater 14 starts heating up the sensor surface 11, and the metal-oxide film 12 starts growing on the sensor surface 11.

After the activation of the gas sensor 1, the gas sensor 1 first operates in the first mode. Step S11 corresponds to operation in the first mode (first step).

In step S11, immediately following the activation of the gas sensor 1, standby air A1 is supplied to the sensor surface 11. Alternatively, standby air A1 may have been being supplied to the sensor surface 11 since before step S11.

FIG. 6 is a diagram illustrating an example of supply of standby air A1 in the gas sensor 1. In an aspect, in step S11, as shown in FIG. 6, standby air A1 may be filtered by activated carbon 61 before being supplied to the sensor surface 11 from the outside of the gas sensor 1 through the flow path F0. Standby air A1 is not necessarily filtered by the activated carbon 61 and may be filtered by any medium including publicly known filtering members used in air filtering. In addition, an electronic apparatus incorporating the gas sensor 1 may include a standby-air-supply system for supplying standby air A1 to the sensor surface 11 as described in an embodiment detailed later.

Thereafter, in the first mode, the gas sensor 1 stands by with standby air A1 being in contact with the sensor surface 11. The control unit 40 switches the operation of the gas sensor 1 from the first mode to the second mode when a prescribed time has elapsed or when the control unit 40 receives a prescribed instruction through the input/output unit 50.

After operating in the first mode, the gas sensor 1 operates in the second mode. Steps S12, S13, S14, and S15 correspond to operation in the second mode (second step).

In step S12, measurement-target air A0 is supplied to the sensor surface 11. Referring to FIG. 1, measurement-target air A0 may be supplied to the sensor surface 11 from the outside of the gas sensor 1 through the flow path F0. In addition, as described in an embodiment detailed later, an electronic apparatus incorporating the gas sensor 1 may include an air blowing unit for supplying measurement-target air A0 to the sensor surface 11.

In step S13, the detection unit 30 detects a change in the resistance value of the metal-oxide film 12. In an aspect, the detection unit 30 may detect a change in the resistance value of the metal-oxide film 12 by sampling the resistance value of the metal-oxide film 12 for a prescribed period.

In step S14, the computation unit 41 computes the quantity of reducing gas in measurement-target air A0 on the basis of results of the detection by the detection unit 30. As the reducing gas comes into contact with the metal-oxide film 12, the resistance value of the metal-oxide film 12 decreases, so that the computation unit 41 can compute the quantity of the reducing gas in accordance with a decrease in the resistance value of the metal-oxide film 12 or a rate of decrease of the resistance value of the metal-oxide film 12.

In step S15, the input/output unit 50 outputs results of the computation by the computation unit 41.

The gas sensor 1 can hence sense reducing gas in measurement-target air A0.

### Embodiment 2

The following will describe Embodiment 2 of the present invention. For convenience of description, members of Embodiment 2 that have the same function as members described in Embodiment 1 will be indicated by the same reference numerals, and description thereof is not repeated. A gas sensor 2 in accordance with the present embodiment performs correction in accordance with the condition of standby air A1.

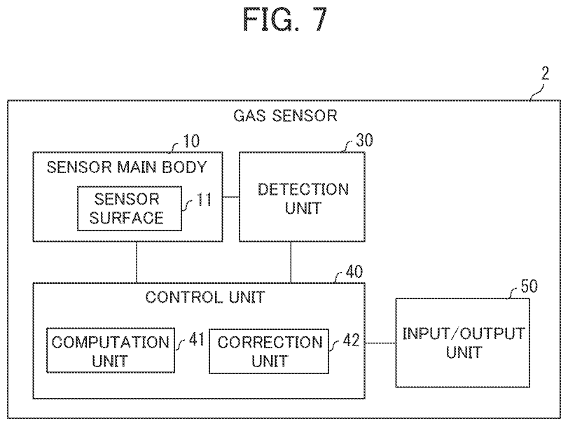

FIG. 7 is a functional block diagram of the gas sensor 2. The gas sensor 2 includes a sensor main body 10, a housing 20, a detection unit 30, a control unit 40, and an input/output unit 50.

In the present embodiment, the control unit 40 includes a correction unit (determination unit) 42 in addition to a computation unit 41. The input/output unit 50 receives inputs on environmental information representing the condition of standby air A1. The condition of standby air A1 may indicate, for example, whether or not standby air A1 has been filtered, what filtering member has been used in filtering standby air A1, or the proportion of the TVOC in standby air A1.

Thereafter, the correction unit 42 determines the condition of standby air A1 on the basis of the inputted environmental information. The computation unit 41 performs correction in accordance with results of the determination by the correction unit 42 in the computation of the quantity of reducing gas. In an aspect, the correction unit 42 may specify a correction coefficient in accordance with the condition of standby air A1 so that the computation unit 41 can compute the quantity of reducing gas using this correction coefficient.

Referring to FIG. 5, the growth of the metal-oxide film 12 on the sensor surface 11 varies depending on the condition of standby air A1. The quantity of reducing gas can be properly computed by the computation unit 41 performing correction in accordance with results of the determination on the condition of standby air A1.

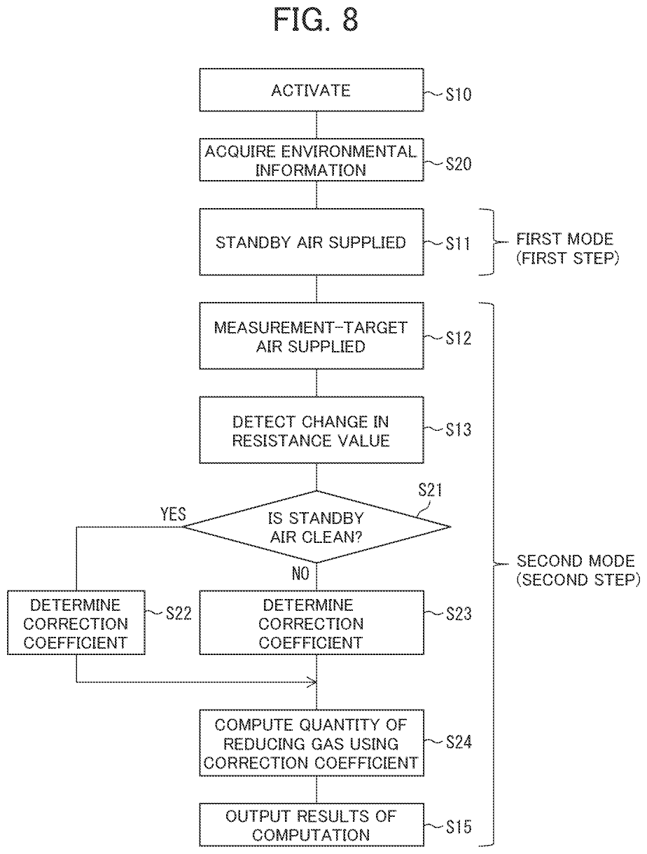

FIG. 8 is a flow chart representing an operation (method of sensing a gas) of the gas sensor 2. The flow chart in FIG. 8 shows additional steps S20 to S24 as well as steps S10, S11, S12, S13, and S15 shown in FIG. 3.

Step S10 is first performed. Step S20 is then performed. In step S20, the correction unit 42 acquires the environmental information inputted to the input/output unit 50.

After step S20, the gas sensor 2 operates in the first mode. Step S11 corresponds to operation in the first mode (first step).

After operating in the first mode, the gas sensor 2 operates in the second mode. Steps S12, S13, S21, S22, S23, S24, and S15 correspond to operation in the second mode (second step).

In the second mode, after steps S12 and S13, step S21 is performed. In step S21, the correction unit 42 determines on the basis of the environmental information whether or not standby air A1 is clean. The correction unit 42 may, for example, determine that standby air A1 is clean if standby air A1 is filtered air and determine that standby air A1 is not clean if standby air A1 is unfiltered air.

Then, if the correction unit 42 determines in step S21 that standby air A1 is clean, the correction unit 42 specifies a correction coefficient corresponding to a clean condition in step S22. On the other hand, if the correction unit 42 determines in step S21 that standby air A1 is not clean, the correction unit 42 specifies a correction coefficient corresponding to a non-clean condition in step S23. Then, in step S24, the computation unit 41 computes the quantity of reducing gas in measurement-target air A0 on the basis of results of the detection in step S13 by the detection unit 30 by using the correction coefficient specified in step S22 or step S23.

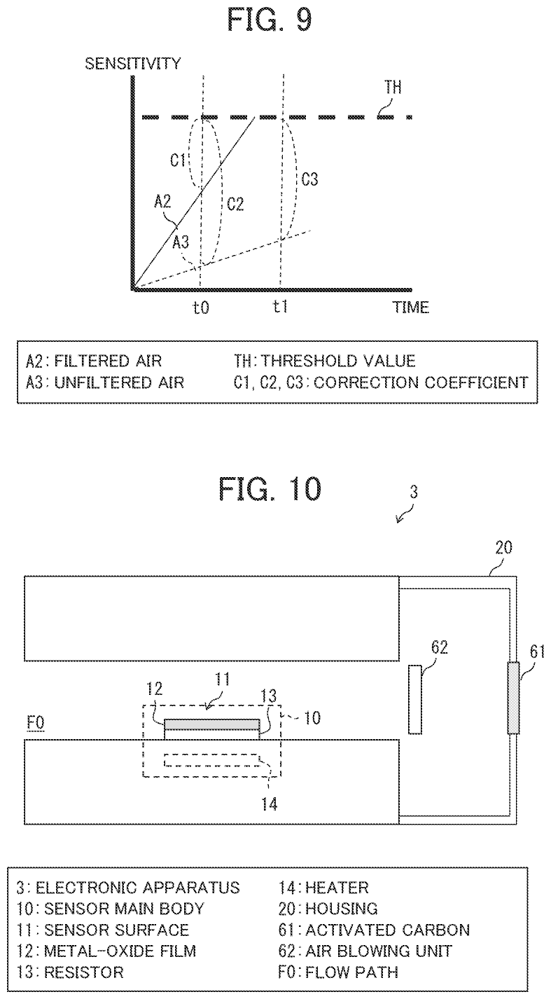

A description is given now of the specification of a correction coefficient in steps S22 and S23. FIG. 9 is a graph representing a relationship between the time elapsed since the gas sensor 2 is activated and a correction coefficient under different aerial conditions.

Referring to FIG. 9, the correction coefficient for correcting the sensitivity of the gas sensor 2 to a prescribed threshold value differs depending on aerial conditions even if the same time has elapsed since the gas sensor 2 is activated. For instance, at time t0, correction coefficient C1 for cases where standby air A1 is filtered air A2 is set to a lower value than correction coefficient C2 for cases where standby air A1 is unfiltered air A3. At time t1 which follows time t0, no correction coefficient is specified if standby air A1 is filtered air A2, and only correction coefficient C3 for cases where unfiltered air A3 is standby air A1 is specified.

In this manner, the correction unit 42 can suitably specify a correction coefficient for correcting the sensitivity of the gas sensor 2 to a prescribed threshold value by specifying a correction coefficient in accordance with the time elapsed after the gas sensor 2 is activated and the condition of standby air A1. Note that the relationship between the time elapsed since the gas sensor 2 is activated and the sensitivity of the gas sensor 2, which is in accordance with the condition of standby air A1, may, for example, be contained in a memory unit (not shown) that is accessible to the control unit 40.

Then, the computation unit 41 can perform correction in the computation of the quantity of reducing gas on the basis of the relationship between the time elapsed since the gas sensor 2 is activated and the sensitivity of the gas sensor 2, which is in accordance with the condition of standby air A1, by computing the quantity of reducing gas using the correction coefficient specified by the correction unit. The computation unit 41 can hence properly compute the quantity of reducing gas.

Alternatively, the correction unit 42 may determine the condition of standby air A1 in further detail on the basis of the environmental information in step S21. In other words, the correction unit 42 may not only determine whether or not standby air A1 is clean, but determine the condition of standby air A1 by three or more levels. Then, the correction unit 42 performs, in place of steps S22 and S23, steps of specifying a correction coefficient for each of the three or more levels, so that the computation unit 41 can perform computation using these specified correction coefficients in step S24. Computation can be hence performed that is better suited to the condition of standby air A1.

### Embodiment 3

The following will describe Embodiment 3 of the present invention. For convenience of description, members of Embodiment 3 that have the same function as members described in Embodiments 1 and 2 will be indicated by the same reference numerals, and description thereof is not repeated. An electronic apparatus 3 in accordance with the present embodiment includes a standby-air-supply system 60 that supplies standby air A1 to the sensor surface 11 in addition to the structural equivalent of the gas sensor 2.

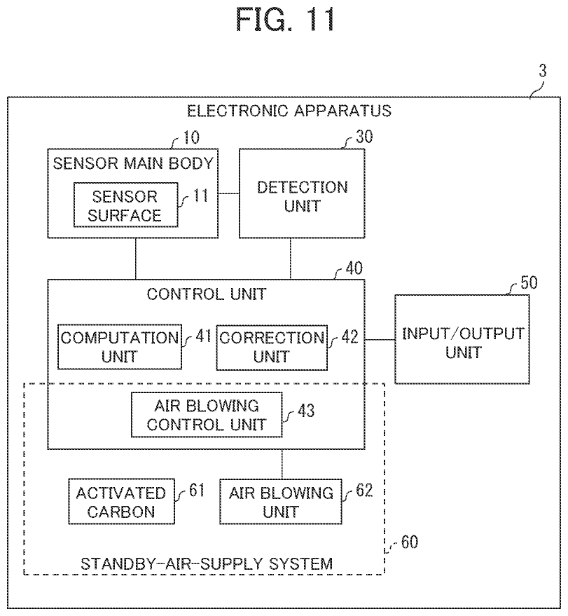

FIG. 10 is a cross-sectional side view of the electronic apparatus 3. FIG. 11 is a functional block diagram of the electronic apparatus 3. The electronic apparatus 3 includes a sensor main body 10, a housing 20, a detection unit 30, a control unit 40, an input/output unit 50, activated carbon 61, and an air blowing unit 62.

In the present embodiment, the control unit 40 includes an air blowing control unit 43 in addition to the computation unit 41 and the correction unit 42. The standby-air-supply system 60 includes the air blowing control unit 43, the activated carbon 61, and the air blowing unit 62 to supply the air filtered by the activated carbon 61 as standby air A1 to the sensor surface 11. This structure successfully enables the air filtered by a filtering member to be supplied to a sensor surface.

The air blowing unit 62 supplies either one or both of measurement-target air A0 and standby air A1 to the sensor surface 11 by blowing air into the flow path F0. The air blowing control unit 43 controls the start/stop of air blowing by the air blowing unit 62 and also controls the air blowing direction. This configuration successfully enables either measurement-target air A0 or standby air A1, which is filtered air, to be supplied to the sensor surface 11.

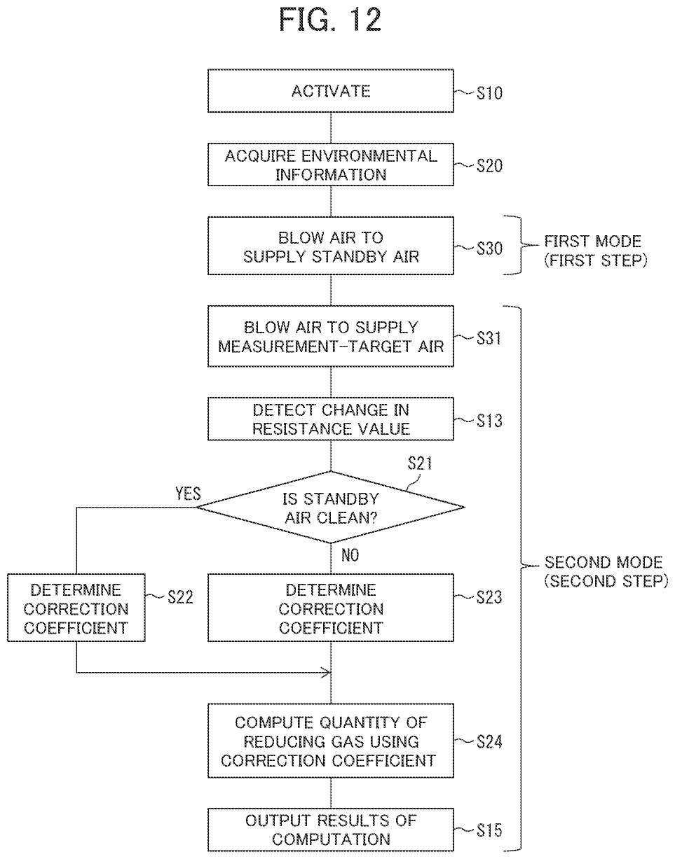

FIG. 12 is a flow chart representing an operation (method of sensing a gas) of the electronic apparatus 3. The flow chart in FIG. 12 shows additional steps S30 and S31 as well as steps S10, S12, S13, and S15 shown in FIG. 3 and steps S20 to S24 shown in FIG. 8.

Steps S10 and S20 are first performed. Then, after step S20, the electronic apparatus 3 operates in the first mode. Step S30 corresponds to operation in the first mode (first step).

In step S30, the air blowing unit 62 blows air in such a manner that standby air A1 generated by the activated carbon 61 is supplied to the sensor surface 11. Standby air A1 is hence supplied to the sensor surface 11. As described earlier, standby air A1 is not necessarily filtered by the activated carbon 61 and may be filtered by any medium including publicly known filtering members used in air filtering such as deodorant beads.

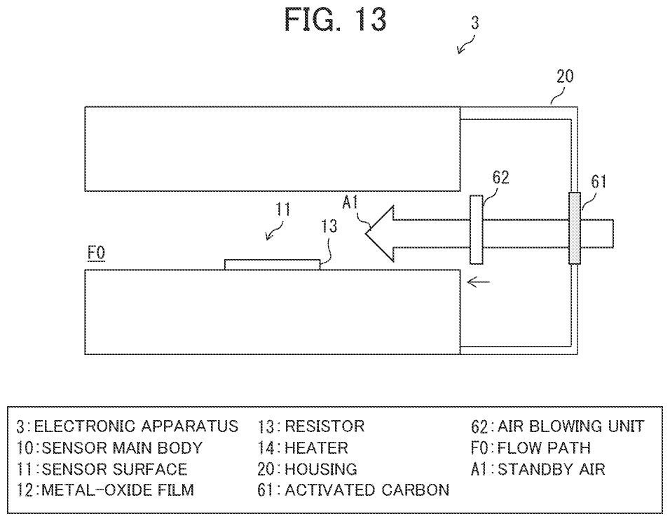

FIG. 13 is a diagram illustrating an example of supply of standby air A1 in the electronic apparatus 3. Referring to FIG. 13, in step S30, standby air A1 generated by the activated carbon 61 can be supplied to the sensor surface 11 by the air blowing unit 62 drawing in air from the outside through the activated carbon 61 and blowing air in such a manner that the air can flow into the flow path F0.

Then, in the first mode, the electronic apparatus 3 stands by with standby air A1 being in contact with the sensor surface 11. The control unit 40 switches the operation of the electronic apparatus 3 from the first mode to the second mode when a prescribed time has elapsed or when the control unit 40 receives a prescribed instruction through the input/output unit 50.

After operating in the first mode, the electronic apparatus 3 operates in the second mode. Steps S31, S13, S21, S22, S23, S24, and S15 correspond to operation in the second mode (second step).

In step S31, the air blowing unit 62 blows air in such a manner that measurement-target air A0 is supplied to the sensor surface 11 without passing through the activated carbon 61. Measurement-target air A0 is hence supplied to the sensor surface 11.

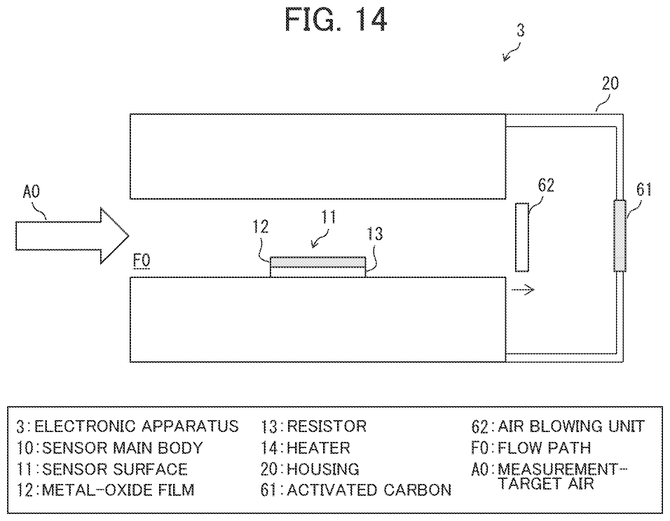

FIG. 14 is a diagram illustrating an example of supply of measurement-target air A0 in the electronic apparatus 3. Referring to FIG. 14, in step S31, the air blowing direction of the air blowing unit 62 is changed from step S30, and the air blowing unit 62 blows air in such a manner that measurement-target air A0 can flow into the flow path F0 without passing through the activated carbon 61. That in turn enables measurement-target air A0 to be supplied to the sensor surface 11.

Then, reducing gas can be sensed by performing steps S13, S21, S22, S23, S24, and S15.

### Embodiment 4

The following will describe Embodiment 4 of the present invention. For convenience of description, members of Embodiment 4 that have the same function as members described in Embodiments 1, 2, and 3 will be indicated by the same reference numerals, and description thereof is not repeated. In an electronic apparatus 4 in accordance with the present embodiment, the standby-air-supply system 60 operates differently from Embodiment 3.

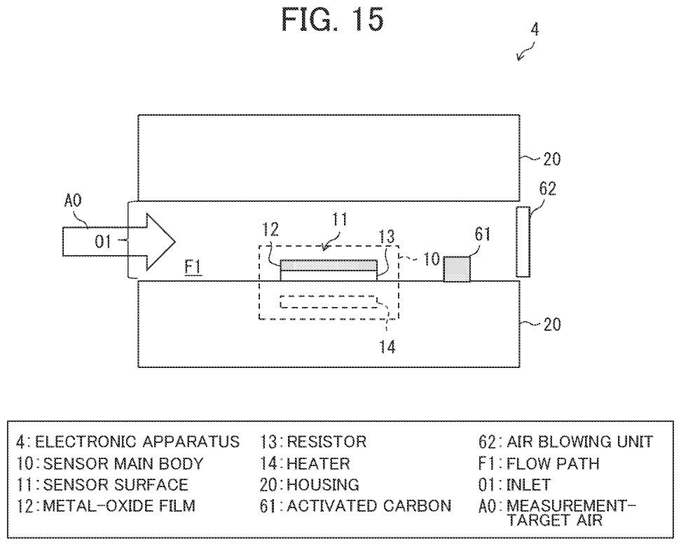

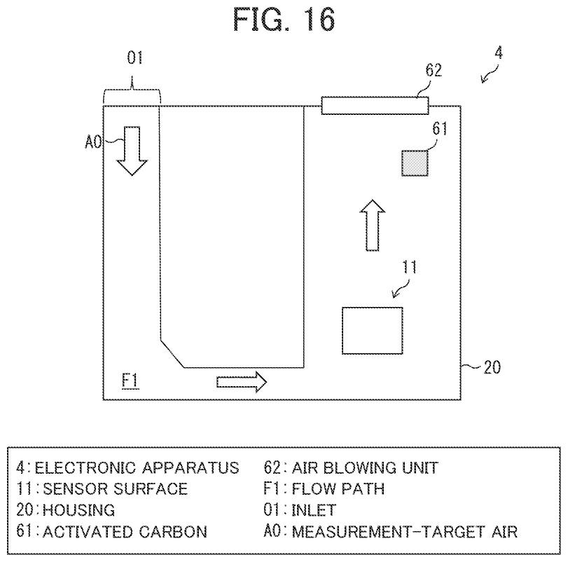

FIG. 15 is a cross-sectional side view of the electronic apparatus 4. FIG. 16 is a cross-sectional top view of the electronic apparatus 4. The electronic apparatus 4 includes a sensor main body 10, a housing 20, a detection unit 30, a control unit 40, an input/output unit 50, activated carbon 61, and an air blowing unit 62. The housing 20 includes a flow path F1 where the sensor surface 11 is provided. The flow path F1 has an inlet 01 through which measurement-target air A0 to be supplied to the sensor surface 11 flows in. The activated carbon 61 is disposed in the flow path F1, downstream of the sensor surface 11 when viewed from the inlet 01.

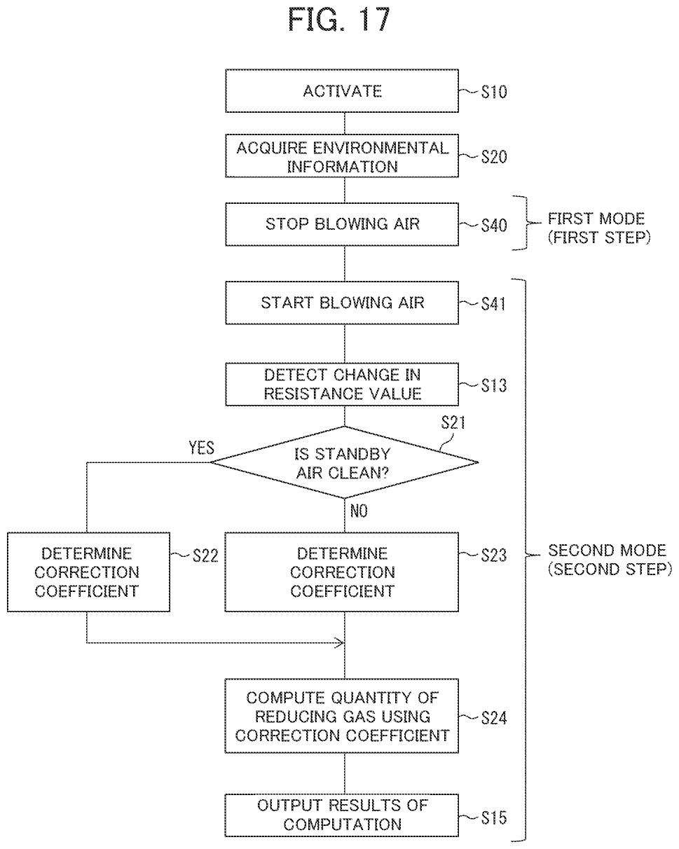

FIG. 17 is a flow chart representing an operation (method of sensing a gas) of the electronic apparatus 4. The flow chart in FIG. 17 shows additional steps S40 and S41 as well as steps S10, S12, S13, and S15 shown in FIG. 3 and steps S20 to S24 shown in FIG. 8.

Steps S10 and S20 are first performed. Then, after step S20, the electronic apparatus 3 operates in the first mode. Step S40 corresponds to operation in the first mode (first step).

In step S40, the air blowing unit 62 does not blow air in the first mode, so that standby air A1 generated by the activated carbon 61 can remain near the sensor surface 11. Standby air A1 is hence supplied to the sensor surface 11. As described earlier, standby air A1 is not necessarily filtered by the activated carbon 61 and may be filtered by any medium including publicly known filtering members used in air filtering.

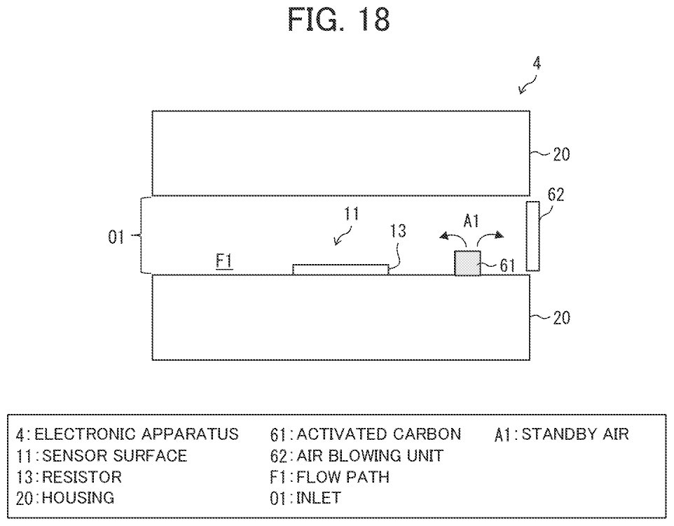

FIG. 18 is a diagram illustrating an example of supply of standby air A1 in the electronic apparatus 4. Referring to FIG. 18, the activated carbon 61, disposed downstream of the sensor surface 11, filters surrounding air to generate standby air A1. Then, in step S40, since the air blowing unit 62 does not blow air, standby air A1 generated by the activated carbon naturally spreads and fills the interior of the flow path F1, thereby remaining near the sensor surface 11. Standby air A1 generated by the activated carbon 61 can be hence supplied to the sensor surface 11.

Then, in the first mode, the electronic apparatus 4 stands by with standby air A1 being in contact with the sensor surface 11. The control unit 40 switches the operation of the electronic apparatus 4 from the first mode to the second mode when a prescribed time has elapsed or when the control unit 40 receives a prescribed instruction through the input/output unit 50.

After operating in the first mode, the electronic apparatus 4 operates in the second mode. Steps S41, S13, S21, S22, S23, S24, and S15 correspond to operation in the second mode (second step).

In step S41, the air blowing unit 62 blows air in such a manner that measurement-target air A0 is supplied to the sensor surface 11 through the inlet 01. Measurement-target air A0 is hence supplied to the sensor surface 11.

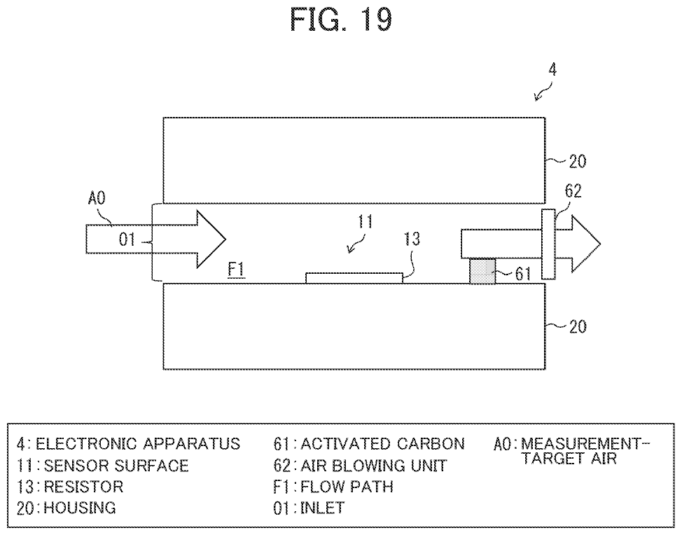

FIG. 19 is a diagram illustrating an example of supply of measurement-target air A0 in the electronic apparatus 4. Referring to FIG. 19, in step S41, the air blowing unit 62 blows air so that measurement-target air A0 can flow into the flow path F1 through the inlet 01. That in turn enables standby air A1 filling the flow path F1 to be pushed out of the interior of the flow path F1 and enables measurement-target air A0 to be supplied to the sensor surface 11. The activated carbon 61, since being disposed downstream of the sensor surface 11, does not filter measurement-target air A0.

Then, reducing gas can be sensed by performing steps S13, S21, S22, S23, S24, and S15.

### Software Implementation

The functions of the gas sensors 1 to 2 and the electronic apparatuses 3 to 4 (hereinafter, the “device(s)”) may be implemented by a program causing a computer to function as the device and causing a computer to function as the control blocks of the device (in particular, each part contained in the control unit 40).

In such cases, the device includes a computer including at least one control device (e.g., a processor) and at least one storage device (e.g., a memory) as hardware that executes the program. The functions described in the embodiments above are implemented by the control and storage devices executing the program.

The program may be stored not temporarily, but in one or more computer-readable storage mediums. The storage medium(s) may or may not be provided in the device. In the latter case, the program may be delivered to the device via any wired or wireless transmission medium.

In addition, the functions of the control blocks may be partially or entirely implemented by logic circuitry. For instance, the scope of the present invention encompasses integrated circuits including logic circuitry that functions as the control blocks.

### General Description

The present invention, in aspect 1 thereof, is directed to a gas sensor including: a sensor surface on which a metal-oxide film grows; a detection unit configured to detect a change in a resistance value of the metal-oxide film; and a computation unit configured to compute a quantity of reducing gas in measurement-target air based on a result of the detection by the detection unit, wherein the gas sensor operates in a first mode in which the gas sensor stands by with standby air, which differs from the measurement-target air, being in contact with the sensor surface immediately after the gas sensor is activated and in a second mode that follows the first mode and in which, the detection unit detects a change in the resistance value, and the computation unit computes the quantity of the reducing gas based on the result of the detection, with the measurement-target air being in contact with the sensor surface.

This structure facilitates the growth of the metal-oxide film on the sensor surface by the gas sensor standing by with the standby air being in contact with the sensor surface immediately after the gas sensor is activated. That in turn improves the post-activation detection sensitivity of the sensor.

In aspect 2 of the present invention, the gas sensor of aspect 1 may be configured such that the standby air is filtered air.

This structure suitably facilitates the growth of the metal-oxide film on the sensor surface because the standby air is filtered air. That in turn improves the post-activation detection sensitivity of the sensor.

In aspect 3 of the present invention, the gas sensor of aspect 1 or 2 may be configured such that the computation unit computes by converting the quantity of the reducing gas to a quantity of carbon dioxide.

This structure improves the post-activation detection sensitivity of the sensor. For this reason, the presence/absence of reducing gas can be readily checked even when the computation is done by converting the quantity of the reducing gas to a quantity of carbon dioxide.

In aspect 4 of the present invention, the gas sensor of any one of aspects 1 to 3 may be configured so as to further include a determination unit configured to determine a condition of the standby air, wherein the computation unit performs correction in accordance with a result of the determination by the determination unit in computing the quantity of the reducing gas.

Since the growth of the metal-oxide film on the sensor surface varies depending on the condition of the standby air, this structure enables suitable computation of the quantity of the reducing gas by performing the correction in accordance with a result of the determination on the condition of the standby air.

In aspect 5 of the present invention, the gas sensor of aspect 4 may be configured such that the computation unit performs the correction based on a relationship between a time elapsed since the gas sensor is activated and sensitivity of the gas sensor in computing the quantity of the reducing gas, the relationship being in accordance with the condition determined by the determination unit.

This structure enables suitable computation of the quantity of the reducing gas by performing the correction on the basis of the relationship between the time elapsed since the gas sensor is activated and the sensitivity of the gas sensor, which is in accordance with the condition of the standby air, in computing the quantity of the reducing gas.

The present invention, in aspect 6 thereof, is directed to an electronic apparatus including: the gas sensor of any of aspects 1 to 5; and a standby-air-supply system including a filtering member configured to generate the standby air by filtering air, the standby-air-supply system being configured to supply the standby air thus generated to the sensor surface.

This structure successfully enables the air filtered by the filtering member to be supplied to the sensor surface by the standby-air-supply system.

In aspect 7 of the present invention, the electronic apparatus of aspect 6 may be configured such that the standby-air-supply system further includes an air blowing unit configured to supply either one or both of the measurement-target air and the standby air to the sensor surface.

Since the standby-air-supply system includes an air blowing unit, this structure successfully enables either measurement-target air or filtered air to be supplied to the sensor surface.

In aspect 8 of the present invention, the electronic apparatus of aspect 7 may be configured such that the air blowing unit blows air in such a manner as to supply the standby air generated by the filtering member to the sensor surface in the first mode and as to supply the measurement-target air to the sensor surface without the measurement-target air having to pass through the filtering member in the second mode.

This structure enables the air blowing unit and the filtering member to supply the standby air to the sensor surface immediately after the gas sensor is activated and thereafter successfully enables the measurement-target air to be supplied to the sensor surface.

In aspect 9 of the present invention, the electronic apparatus of aspect 7 may be configured such that the gas sensor has a flow path where the sensor surface is disposed, the flow path has an inlet into which the measurement-target air to be supplied to the sensor surface flows, the filtering member is disposed in the flow path, downstream of the sensor surface when viewed from the inlet, and the air blowing unit does not blow air in the first mode to have the standby air generated by the filtering member remain near the sensor surface and blows air in the second mode in such a manner that the measurement-target air flows into the flow path through the inlet, to supply the measurement-target air to the sensor surface.

This structure enables the air blowing unit and the filtering member to supply the standby air to the sensor surface immediately after the gas sensor is activated and thereafter successfully enables the measurement-target air to be supplied to the sensor surface.

The present invention, in aspect 10 thereof, is directed to a method of sensing a gas by a gas sensor including a sensor surface on which a metal-oxide film is grown, the method including: a first step of the gas sensor standing by with standby air, which differs from measurement-target air, being in contact with the sensor surface immediately after the gas sensor is activated; and after the first step, a second step of the gas sensor detecting a change in a resistance value of the metal-oxide film and computing a quantity of reducing gas in the measurement-target air based on a result of the detection, with the measurement-target air being in contact with the sensor surface.

This method achieves the same advantages as aspect 1.

The present invention is not limited to the description of the embodiments above and may be altered within the scope of the claims. Embodiments based on a proper combination of technical means disclosed in different embodiments are encompassed in the technical scope of the present invention. Furthermore, new technological features can be created by combining different technical means disclosed in the embodiments.

While there have been described what are at present considered to be certain embodiments of the invention, it will be understood that various modifications may be made thereto, and it is intended that the appended claim cover all such modifications as fall within the true spirit and scope of the invention.

What is claimed is:

1. A gas sensor comprising:
a sensor surface on which a metal-oxide film grows;
a detection unit configured to detect a change in a resistance value of the metal-oxide film; and
a computation unit configured to compute a quantity of reducing gas in measurement-target air based on a result of the detection by the detection unit, wherein
the gas sensor operates in a first mode in which the gas sensor stands by with standby air, which differs from the measurement-target air, being in contact with the sensor surface immediately after the gas sensor is activated and in a second mode that follows the first mode and in which, the detection unit detects a change in the resistance value, and the computation unit computes the quantity of the reducing gas based on the result of the detection, with the measurement-target air being in contact with the sensor surface.

2. The gas sensor according to claim 1, wherein the standby air is filtered air.

3. The gas sensor according to claim 1, wherein the computation unit computes by converting the quantity of the reducing gas to a quantity of carbon dioxide.

4. The gas sensor according to claim 1, further comprising a determination unit configured to determine a condition of the standby air, wherein the computation unit performs correction in accordance with a result of the determination by the determination unit in computing the quantity of the reducing gas.

5. The gas sensor according to claim 4, wherein the computation unit performs the correction based on a relationship between a time elapsed since the gas sensor is activated and sensitivity of the gas sensor in computing the quantity of the reducing gas, the relationship being in accordance with the condition determined by the determination unit.

6. An electronic apparatus comprising:
the gas sensor according to claim 1; and
a standby-air-supply system comprising a filtering member configured to generate the standby air by filtering air, the standby-air-supply system being configured to supply the standby air thus generated to the sensor surface.

7. The electronic apparatus according to claim 6, wherein the standby-air-supply system further comprises an air blowing unit configured to supply either one or both of the measurement-target air and the standby air to the sensor surface.

8. The electronic apparatus according to claim 7, wherein the air blowing unit blows air in such a manner as to supply the standby air generated by the filtering member to the sensor surface in the first mode and as to supply the measurement-target air to the sensor surface without the measurement-target air having to pass through the filtering member in the second mode.

9. The electronic apparatus according to claim 7, wherein
the gas sensor has a flow path where the sensor surface is disposed,
the flow path has an inlet into which the measurement-target air to be supplied to the sensor surface flows,
the filtering member is disposed in the flow path, downstream of the sensor surface when viewed from the inlet, and
the air blowing unit does not blow air in the first mode to have the standby air generated by the filtering member remain near the sensor surface and blows air in the second mode in such a manner that the measurement-target air flows into the flow path through the inlet, to supply the measurement-target air to the sensor surface.

10. A method of sensing a gas by a gas sensor including a sensor surface on which a metal-oxide film is grown, the method comprising:
a first step of the gas sensor standing by with standby air, which differs from measurement-target air, being in contact with the sensor surface immediately after the gas sensor is activated; and
after the first step, a second step of the gas sensor detecting a change in a resistance value of the metal-oxide film and computing a quantity of reducing gas in the measurement-target air based on a result of the detection, with the measurement-target air being in contact with the sensor surface.
